(19) United States
(12) Patent Application Publication
(10) Pub. No.: US 2026/0260731 A1
(43) Pub. Date: Sep. 3, 2026

(54) METHODS, SYSTEMS, AND MEDIA FOR PERFORMING COGNITIVE-BASED THERAPY

(71) Applicant: Vijay Ramamoorthy, Sacramento, CA (US)

(72) Inventor: Vijay Ramamoorthy, Sacramento, CA (US)

(21) Appl. No.: 19/659,321

(22) Filed: Apr. 27, 2026

Related U.S. Application Data
(63) Continuation of application No. 18/735,596, filed on Jun. 6, 2024, now abandoned.

(51) Int. Cl. G16H 20/70; G06F 40/253; G16H 80/00

(52) U.S. Cl. CPC G16H 20/70; G06F 40/253; G16H 80/00

(57) ABSTRACT

A method for performing cognitive-based therapy, comprising: performing a process to be emulated, the process including: uttering a first single independent clause identifying a first feature in a target subject matter; drawing a first symbol after uttering the first single independent clause; uttering a second single independent clause identifying a second feature in the target subject matter; drawing a second symbol proximate to the first symbol and along a first non-intersecting path after uttering the second single independent clause; uttering a third single independent clause identifying a third feature in the target subject matter; drawing a third symbol proximate to the second symbol and along the first non-intersecting path after uttering the third single independent clause; instructing a client to identify first features in a target subject matter by emulating the process; instructing the client to draw all symbols along a second non-intersecting path before the client emulates the process.

### CROSS-REFERENCE TO RELATED APPLICATIONS

The present application claims priority to U.S. Nonprovisional Application No. Ser. No. 18/735,596 filed Jun. 6, 2024, titled “METHODS, SYSTEMS, AND MEDIA FOR PERFORMING COGNITIVE-BASED THERAPY,” which is hereby incorporated by reference.

### TECHNICAL FIELD

Embodiments disclosed herein can relate to methods, media, and systems for performing cognitive-based therapy.

### BACKGROUND

In general, some people may have difficulty making decisions, solving problems, and thinking through emotional issues.

There is a need for a method for performing cognitive-based therapy that can assist people with making decisions, solving problems, and thinking through emotional issues, etc. by instructing or teaching them to draw a diagram that includes readable symbols. As a person draws symbols along a prescribed path, the person is better able to sequence, structure, follow and continue their line thought. They are also better able to focus and express their thoughts. The person is also able to read symbols in the diagram to remember any thoughts they had while drawing the symbols in the diagram. The symbols may form representations of the person's thoughts.

In some embodiments, the process can be performed in an instructive capacity and, if the client is familiar with the process, the process can be performed in a facilitative capacity to guide the client in refining the process.

In some embodiments, the process can be used to help people with conditions like attention deficit disorder (ADHD) and/or traumatic brain injuries.

### SUMMARY

This summary is provided to introduce a variety of concepts and/or aspects in a simplified form that is further disclosed in the detailed description, below. This summary is not intended to identify key or essential inventive concepts of the claimed subject matter, nor is it intended for determining the scope of the claimed subject matter.

In some embodiments, a method for performing cognitive-based therapy can include performing, by a coach, a process to be emulated, the process including: uttering a first single independent clause identifying a first feature in a target subject matter; drawing a first symbol after uttering the first single independent clause; uttering a second single independent clause identifying a second feature in the target subject matter; drawing a second symbol proximate to the first symbol and along a first non-intersecting path after uttering the second single independent clause; uttering a third single independent clause identifying a third feature in the target subject matter; drawing a third symbol proximate to the second symbol and along the first non-intersecting path after uttering the third single independent clause; instructing, by the coach, a client to identify first features in a target subject matter by emulating the process; instructing, by the coach, the client to draw all symbols along a second non intersecting path before the client emulates the process.

### BRIEF DESCRIPTION OF THE DRAWINGS

A complete understanding of the present features or aspects and the advantages and features thereof will be more readily understood by reference to the following detailed description when considered in conjunction with the accompanying drawings wherein:

FIG. 1A illustrates a block diagram of a system for performing cognitive-based therapy, according to some embodiments disclosed herein;

FIG. 1B illustrates a simplified diagram of a coach instructing or guiding a client, according to some embodiments disclosed herein;

FIG. 2A illustrates a flowchart of a method for performing cognitive-based therapy, according to some embodiments disclosed herein;

FIG. 2B illustrates a flowchart of a process to be emulated, according to some embodiments disclosed herein;

FIG. 2C illustrates a flowchart of a method for performing cognitive-based therapy, according to some embodiments disclosed herein;

FIG. 2D illustrates a flowchart of a process to be emulated, according to some embodiments disclosed herein;

FIG. 3A illustrates at least one image that can be presented on a display, according to some embodiments disclosed herein;

FIG. 3B illustrates a pattern of symbols drawn on a suitable medium, according to some embodiments disclosed herein;

FIG. 3C illustrates a method of placing blocks on a surface, according to some embodiments disclosed herein;

FIG. 4A illustrates a flowchart of a method for performing cognitive-based therapy, according to some embodiments disclosed herein;

FIG. 4B illustrates a block diagram of a system for performing cognitive-based therapy including a computer-readable medium, according to some embodiments disclosed herein;

FIG. 4C illustrates a user interface for generating symbols and text output, according to some embodiments disclosed herein; and

FIG. 5 illustrates a block diagram of a computing device for performing cognitive based therapy, according to some embodiments disclosed herein.

The drawings are not necessarily to scale, and certain features and certain views of the drawings may be shown exaggerated in scale or in schematic in the interest of clarity and conciseness.

### DETAILED DESCRIPTION

Any specific details of features or aspects are used for demonstration purposes only, and no unnecessary limitations or inferences are to be understood therefrom.

Before describing in detail exemplary aspects, it is noted that the aspects reside primarily in combinations of components and procedures related to the systems, methods, and media disclosed herein. Accordingly, the systems, methods, and media components and processes have been represented where appropriate by conventional symbols in the drawings, showing only those specific details that are pertinent to understanding the aspects of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship, or order between such entities or elements. Furthermore, there is no intention to be bound by any expressed or implied theory presented in the preceding technical field, background, summary, or the following detailed description. It is also to be understood that the specific devices and processes illustrated in the attached drawings, and described in the following specification, are simply exemplary aspects of the inventive concepts defined in the appended claims. Hence, specific steps, process order, dimensions, component connections, and other physical characteristics relating to the aspects disclosed herein are not to be considered as limiting, unless the claims expressly state otherwise. The use or mention of any single element contemplates a plurality of such element, and the use or mention of a plurality of any element contemplates a single element (for example, “a device” and “devices” and “a plurality of devices” and “one or more devices” and “at least one device” contemplate each other), regardless of whether particular variations are identified and/or described, unless impractical, impossible, or explicitly limited.

Mechanisms (which can include systems, methods, media, or any combination thereof), for performing cognitive-based therapy are disclosed herein. In some embodiments, the methods can be performed by a coach. The coach can include at least one person that guides or teaches a client how to perform any method, process, or subprocess disclosed herein. The client can include at least one other person that receives guidance or instruction from the coach.

The methods disclosed herein can be therapeutic for the client. As the client performs the methods disclosed herein, the client is better able to focus and express their thoughts in a more coherent manner. The client makes a pattern of symbols or blocks along a non intersecting path while expressing their thoughts as guided or instructed by the coach. After the client makes the pattern of symbols or blocks, the client is better able to read the pattern and recollect the thoughts and feelings they had when they drew the pattern of symbols or placed the pattern blocks on a suitable surface.

In some embodiments, the symbols can include any suitable symbols such as, for example, dots (e.g., circular symbols) or shapes (e.g., rectangles, squares, circles, etc.). In some embodiments, the symbols are not letters or characters of a language. In some embodiments, the symbols are not numbers. In some embodiments, the blocks can be made of any suitable durable material such as, for example, wood, plastic, metal, or any combination thereof.

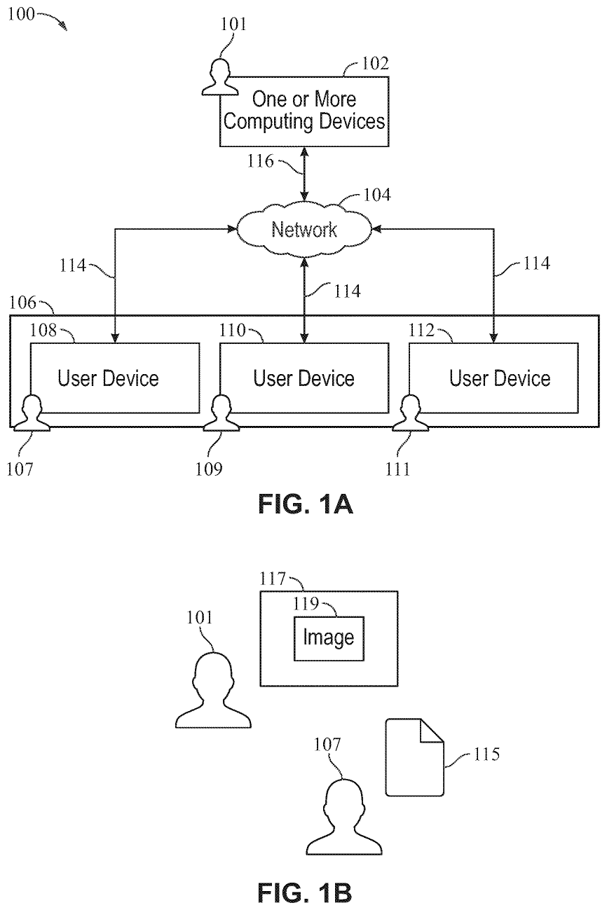

Referring to FIG. 1A, a system 100 for performing cognitive-based therapy can be used in some embodiments disclosed herein. In some embodiments, system 100 can comprise one or more computing devices 102, a network 104 (e.g., communication network), one or more user devices 106, or any combination thereof. In some embodiments, the one or more user devices 106 can include a first user device 108, a second user device 110, a third user device 112, any other user device(s), or any combination thereof. In some embodiments, the system 100 can be configured to perform any method, process, or subprocess disclosed herein.

The one or more computing devices 102 can be any suitable computing device(s) for storing data, programs, or a combination thereof, for performing cognitive-based therapy. In some embodiments, the one or more computing devices 102 can be configured to generate and send any video recording(s), video stream(s), audio recording(s), audio stream(s), or any combination thereof, to any of the one or more user devices 106. In some embodiments, at least one coach 101 operating the one or more computing devices 102 can guide or instruct at least one client 107, 109, 111 via the video recording(s), video stream(s), audio recording(s), audio stream(s), or any combination thereof. The one or more computing devices 102 can include a mobile device, such as a mobile phone, a tablet computer, a wearable computer, a laptop computer, a vehicle (e.g., a car, a boat, an airplane, or any other suitable vehicle), any other suitable mobile device, any suitable non-mobile device (e.g., a desktop computer, entertainment system, etc.), or any combination thereof. As another example, the one or more computing devices 102 can include a media playback device, such as a television, a projector device, a game device or game console, any other suitable computing device, or any combination thereof.

In some embodiments, the one or more user devices 106 can include any suitable computing device(s) for storing data, programs, or a combination thereof, for performing cognitive-based therapy. In some embodiments, the one or more user devices 106 can be configured to receive any video recording(s), video stream(s), audio recording(s), audio stream(s), or any combination thereof, from the one or more computing devices 102.

The network 104 can include a wired network, a wireless network, or a combination thereof. In some embodiments, the network 104 can include the Internet, an intranet, a wide area network (WAN), a local-area network (LAN), a digital subscriber line (DSL) network, a frame relay network, an asynchronous transfer mode (ATM) network, a virtual private network (VPN), any other suitable communication network, or any combination thereof. In some embodiments, one or more communications links 114 can connect the one or more user devices 106 to the network 104. In some embodiments, one or more communication links 116 can connect the network 104 to the one or more computing devices 102. The one or more communication links 114, 116 can be any communication links suitable for communicating information between the one or more user devices 106 and the one or more computing devices 102, such as, for example, network links, dial-up links, wireless links, hard-wired links, any other suitable communications links, or any combination thereof.

While the one or more computing devices 102 are illustrated as one device, any suitable number of computing devices can be included in the one or more computing devices 102 in some embodiments.

While three user devices 108, 110, 112 are illustrated in FIG. 1A to avoid over complicating the figure, any suitable number of computing devices can be included in the one or more user devices 106 in some embodiments.

In some embodiments, the one or more computing devices 102 and the one or more user devices 106 can be implemented using any suitable hardware. For example, any device of the one or more computing devices 102 and the one or more user devices 106 can be implemented using any suitable general-purpose computer or special-purpose computer. Any general-purpose computer or special-purpose computer can include any suitable hardware.

Referring to FIG. 1B, the at least one coach 101 can use a display 117 to present at least one image 119 for guiding and instructing at least one client 107 to perform the methods and processes disclosed herein. In some embodiments, the at least one coach 101 can guide and instruct the at least one client 107 in person or virtually, using the system 100 in FIG. 1A. The client 107 can draw any suitable patterns and symbols on any suitable medium such as, for example, paper 115.

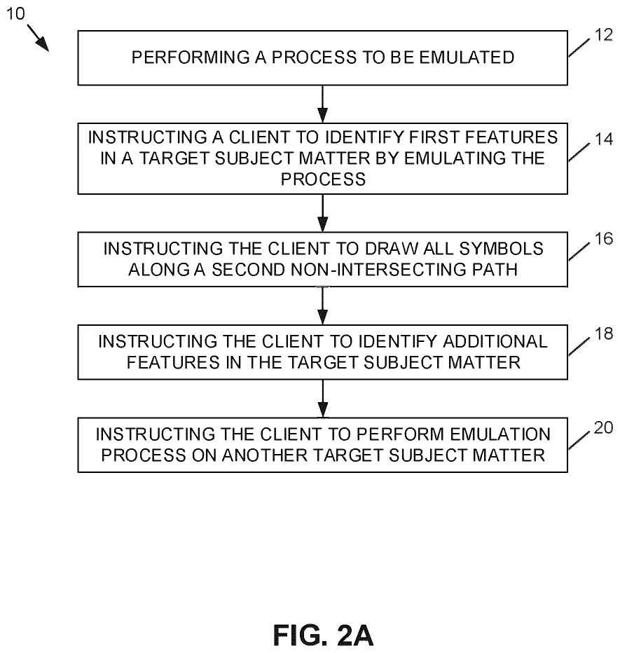

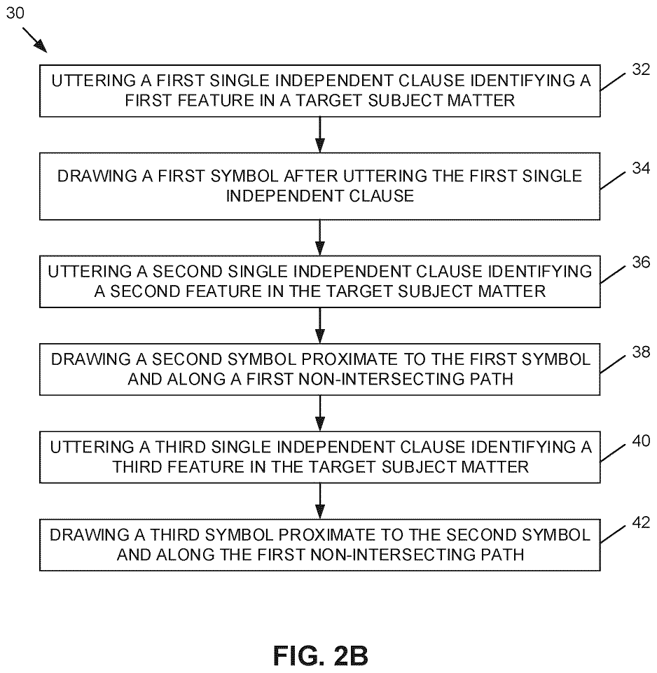

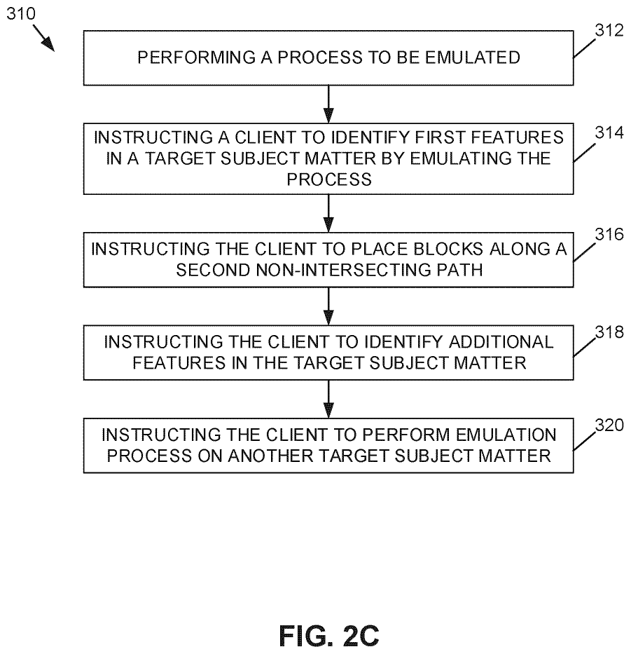

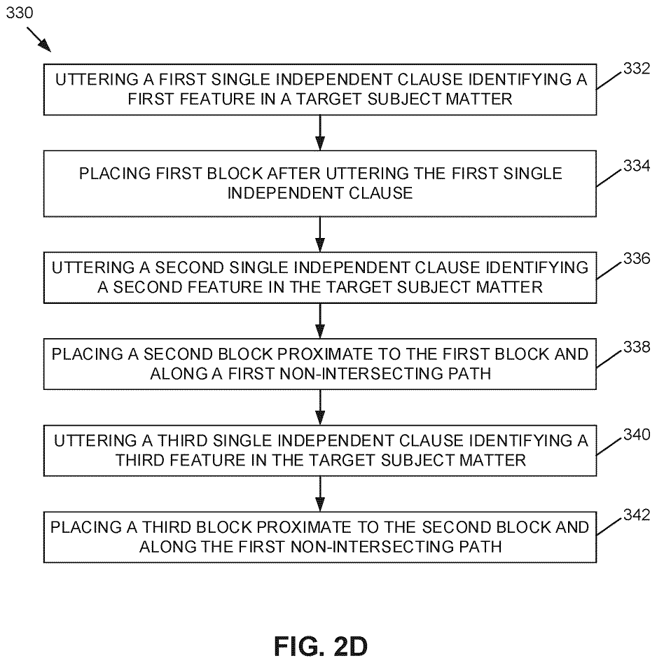

Referring to FIG. 2A, a flowchart of a method 10 for performing cognitive-based therapy is illustrated. In some embodiments, the method 10 can include performing 12 a process to be emulated. Referring to FIG. 2B, a flowchart of a process 30 to be emulated is illustrated. At least one coach such as coach 101 in FIG. 1A can perform the process 30 to be emulated. The process 30 can be emulated by at least one client (e.g., 107, 109, 111 in FIG. 1A). The process 30 to be emulated can include uttering 32, by at least one coach 101, a first single independent clause identifying a first feature in a target subject matter. In some embodiments target subject matter can include an image (e.g., 119 in FIG. 1B), a story, an event, etc.

In some embodiments, the process 30 can include drawing 34 a first symbol after uttering 32 the first single independent clause. In some embodiments, the process 30 can include uttering 36 a second single independent clause identifying a second feature in the target subject matter. In some embodiments, the process 30 can include drawing 38 a second symbol proximate to the first symbol and along a first non-intersecting path after uttering 36 the second single independent clause. In some embodiments, the process 30 can include uttering 40 a third single independent clause identifying a third feature in the target subject matter. In some embodiments, the process 30 can include drawing 42 a third symbol proximate to the second symbol and along the first non-intersecting path after uttering 40 the third single independent clause. The process 30 can include drawing any additional symbols along the first non-intersecting path so that neighboring symbols are proximate to each other. For example, neighboring symbols can be drawn within a predetermined distance from each other.

Referring back to FIG. 2A, in some embodiments, the method 10 can include instructing 14 a client to identify first features in a target subject matter by emulating the process 30. In some embodiments, the method 10 can include instructing 16 the client to draw all symbols along a second non-intersecting path before the client emulates the process 30. In some embodiments, the method 10 can include instructing 18 the client to identify additional features in the target subject matter by uttering. In some embodiments, the method 10 can include instructing 20 the client to perform the process to be emulated using another target subject matter.

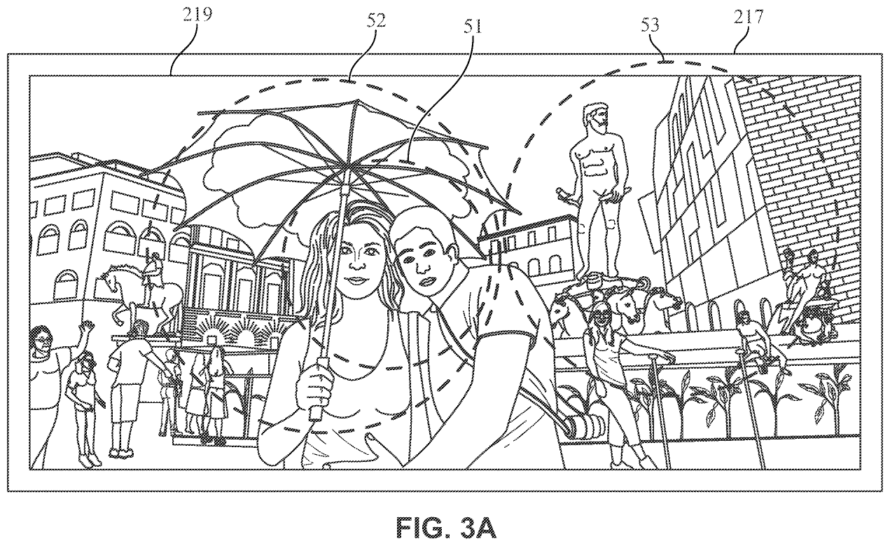

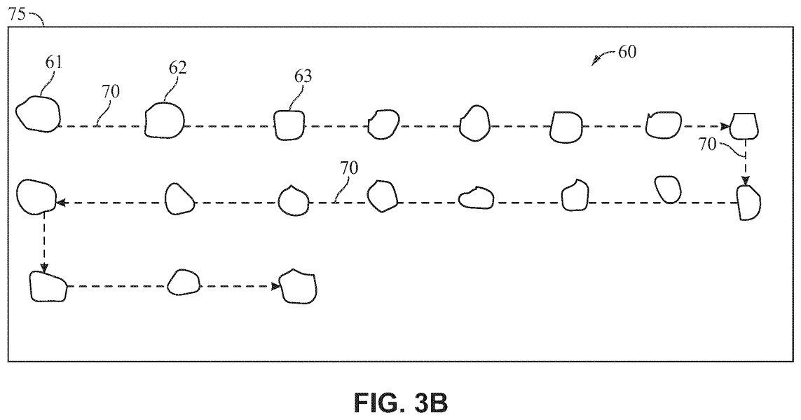

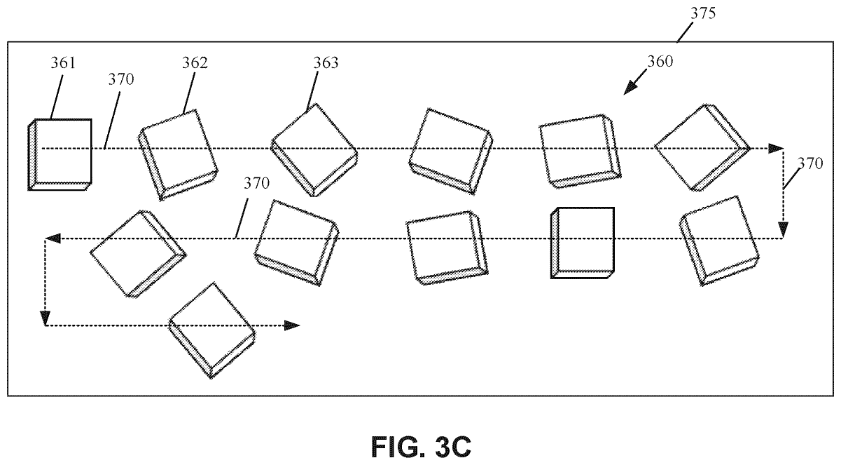

Referring to FIG. 3A, in some embodiments, at least one image 219 can be presented on a display 217. In some embodiments, a user device (e.g., 106, 108, 110 in FIG. IA), the one or more computing devices 102 in FIG. IA, or a combination thereof can include a display such as the display 217. In some embodiments, at least one coach (e.g., 101 in FIG. IA) can perform the process 30 to be emulated while the at least one image 219 is presented.

In some embodiments, the at least one coach 101 can utter a first single independent clause identifying a first feature 51 in a target subject matter such as the image 219. For example, the first single independent clause identifying the first feature 51 in the target subject matter can be “a couple is posing for a picture.” Referring to FIG. 3B, after uttering the first single independent clause, the at least one coach 101 can draw a first symbol 61.

Referring back to FIG. 3A, after drawing the first symbol 61, the at least one coach 101 can utter a second single independent clause identifying a second feature 52 in the target subject matter. For example, the second single independent clause identifying the second feature 52 in the target subject matter can be “the woman is holding an umbrella.” Referring back to FIG. 3B, after uttering the second single independent clause, the at least one coach 101 can draw a second symbol 62 proximate to the first symbol 61 and along a first non intersecting path 70.

Referring back to FIG. 3A, after drawing the second symbol 62 proximate to the first symbol 61, the at least one coach 101 can utter a third single independent clause identifying a third feature 53 in the target subject matter. For example, the third single independent clause identifying the third feature 53 in the target subject matter can be “statues are positioned behind the couple.” Referring back to FIG. 3B, after uttering the third single independent clause identifying the third feature 53 in the target subject matter, the at least one coach 101 can draw a third symbol 63 proximate to the second symbol 62 and along the first non intersecting path 70.

The person (e. g, at least one coach 101 in FIG. 1A, the at least one client 107 in FIG. 1A) can better recollect each of their utterances by associating each drawn symbol with a corresponding utterance. In some embodiments, each symbol of the plurality of symbols 60 can be irregularly drawn which helps the person recollect a corresponding utterance made before each symbol was drawn by looking at the symbol drawn. The plurality of symbols 60 can therefore be readable by the person that performed or emulated the process 30 to be emulated.

The at least one coach 101 can instruct at least one client 107 to emulate the process 30 to be emulated. The at least one client 107 can emulate the process 30 by performing the process 30. For example, the at least one client 107 can utter single independent clauses identifying additional features in the at least one image 217 or any additional images, and draw symbols along a non-intersecting path. In some embodiments, the at least one client can identify features in another target subject matter. While the target subject matter is shown as being the image 219, the target subject matter can be a video, a story, an event, an image, etc., or any combination thereof.

In some embodiments, the symbols 61, 62, 63 can be included in a plurality of symbols 60 that is drawn on a medium 75. The medium 75 can include any suitable medium such as, for example, paper (e.g., 115 in FIG. 1B) or a display (e.g., 117 in FIG. 1B, 217 in FIG. 3A).

Referring to FIG. 2C, a flowchart of a method 310 for performing cognitive-based therapy is illustrated. In some embodiments, the method 310 can include performing 312 a process to be emulated. Referring to FIG. 2D, a flowchart of a process 330 to be emulated is illustrated. At least one coach such as coach 101 in FIG. 1A can perform the process 330 to be emulated. The process 330 can be emulated by at least one client (e.g., 107, 109, 111 in FIG. 1A). The process 330 to be emulated can include uttering 332, by at least one coach 101, a first single independent clause identifying a first feature in a target subject matter. In some embodiments, the target subject matter can include an image (e.g., 119 in FIG. 1B), a story, an event, etc.

In some embodiments, the process 330 can include placing 334 a first block on any suitable surface after uttering 332 the first single independent clause. In some embodiments, the process 330 can include uttering 336 a second single independent clause identifying a second feature in the target subject matter. In some embodiments, the process 330 can include placing 338 a second block proximate to the first block on the surface and along a first non intersecting path on the surface after uttering 336 the second single independent clause. In some embodiments, the process 330 can include uttering 340 a third single independent clause identifying a third feature in the target subject matter. In some embodiments, the process 330 can include placing 342 a third block proximate to the second block on the surface and along the first non-intersecting path on the surface after uttering 340 the third single independent clause. The process 330 can include placing any additional blocks on the surface and along the first non-intersecting path on the surface so that neighboring blocks are proximate to each other. For example, neighboring blocks can be placed on the surface within a predetermined distance from each other.

Referring back to FIG. 2C, in some embodiments, the method 310 can include instructing 314 a client to identify first features in a target subject matter by emulating the process 330. In some embodiments, the method 310 can include instructing 316 the client to place all blocks on a surface and along a second non-intersecting path on the surface before the client emulates the process 330. In some embodiments, the method 310 can include instructing 318 the client to identify additional features in the target subject matter by uttering. In some embodiments, the method 310 can include instructing 320 the client to perform the process 330 to be emulated using another target subject matter.

Referring to FIG. 3A, in some embodiments, at least one image 219 can be presented on a display 217. In some embodiments, a user device (e.g., 106, 108, 110 in FIG. 1A), the one or more computing devices 102 in FIG. 1A, or a combination thereof can include a display such as the display 217. In some embodiments, at least one coach (e.g., 101 in FIG. 1A) can perform the process 330 to be emulated while the at least one image 219 is presented.

In some embodiments, the at least one coach 101 can utter a first single independent clause identifying a first feature 51 in a target subject matter such as the image 219. For example, the first single independent clause identifying the first feature 51 in the target subject matter can be “a couple is posing for a picture.” Referring to FIG. 3C, after uttering the first single independent clause, the at least one coach 101 can place a first block 361 on a surface 375.

Referring back to FIG. 3A, after placing the first block 361 on the surface 375, the at least one coach 101 can utter a second single independent clause identifying a second feature 52 in the target subject matter. For example, the second single independent clause identifying the second feature 52 in the target subject matter can be “the woman is holding an umbrella.” Referring back to FIG. 3C, after uttering the second single independent clause, the at least one coach 101 can place a second block 362 on the surface 375 proximate to the first block 361 on the surface 375 and along a first non-intersecting path 370 on the surface 375.

Referring back to FIG. 3A, after placing the second block 362 proximate to the first block 361 on the surface 375, the at least one coach 101 can utter a third single independent clause identifying a third feature 53 in the target subject matter. For example, the third single independent clause identifying the third feature 53 in the target subject matter can be “statues are positioned behind the couple.” Referring back to FIG. 3C, after uttering the third single independent clause identifying the third feature 53 in the target subject matter, the at least one coach 101 can place a third block 363 on the surface 375 proximate to the second block 362 on the surface 375 and along the first non-intersecting path 370 on the surface 375.

The person (e. g, at least one coach 101 in FIG. 1A, the at least one client 107 in FIG. 1A) can better recollect each of their utterances by associating each placed block with a corresponding utterance. In some embodiments, each block of the plurality of blocks 360 can be irregularly placed which helps the person recollect a corresponding utterance made before each block was placed on the surface 375 by looking at the orientation of the placed block. The plurality of blocks 360 can therefore be readable by the person that performed or emulated the process 330 to be emulated.

The at least one coach 101 can instruct at least one client 107 to emulate the process 330 to be emulated. The at least one client 107 can emulate the process 330 by performing the process 330. For example, the at least one client 107 can utter single independent clauses identifying additional features in the at least one image 217 or any additional images, and place blocks along a non-intersecting path on a surface. In some embodiments, the at least one client can identify features in another target subject matter. While the target subject matter is shown as being the image 219, the target subject matter can be a video, a story, an event, an image, etc., or any combination thereof.

In some embodiments, the blocks 361, 362, 363 can be included in a plurality of blocks 360 can be placed on any suitable surface 375 such as, for example, a table surface, a ground surface, a floor surface, a furniture surface, etc.

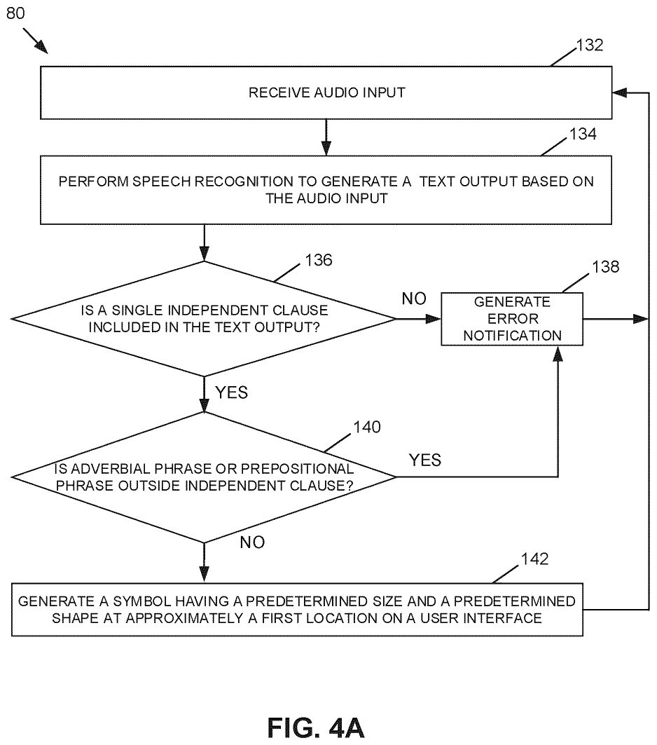

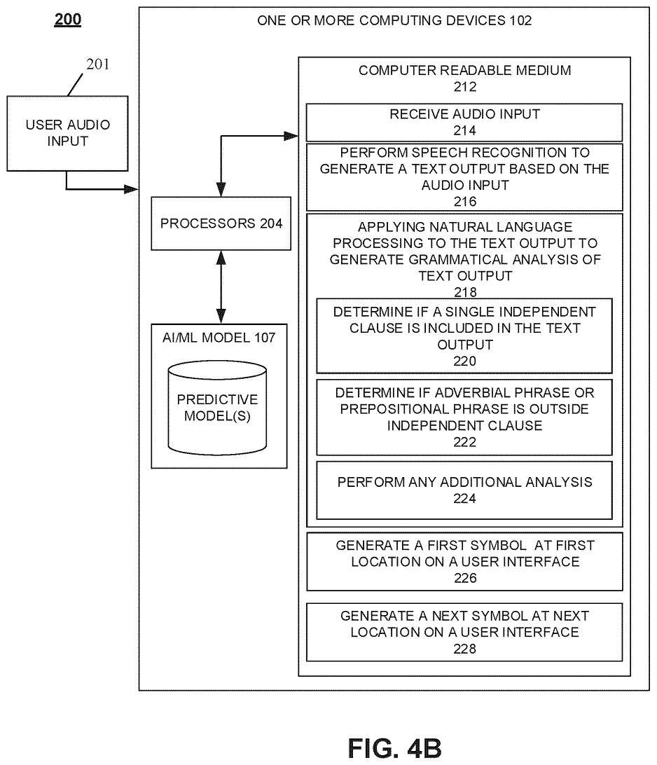

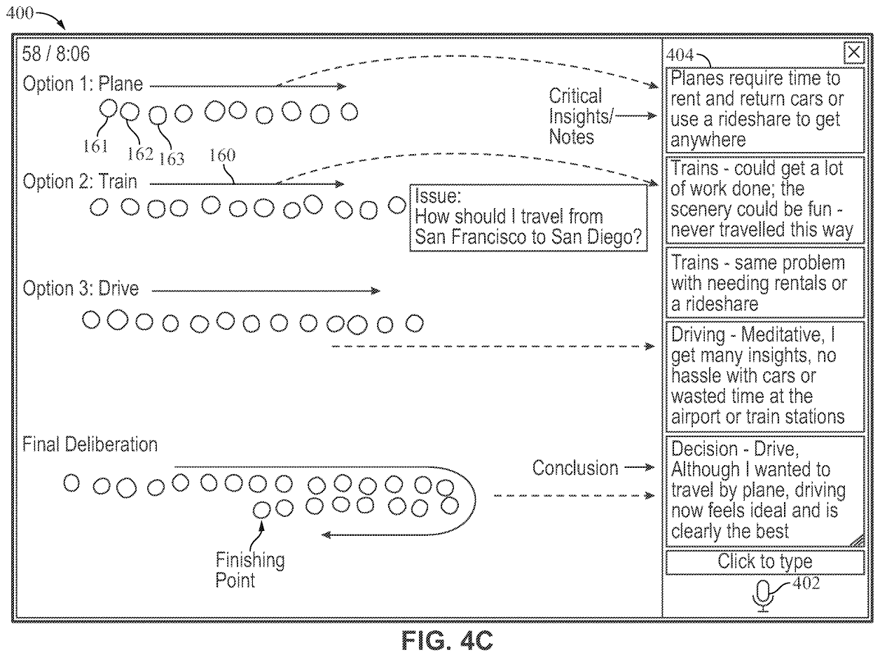

Referring to FIG. 4A, a flowchart of a process 80 for performing cognitive-based therapy is shown. In some embodiments, the process 80 can be a computer-implemented process 80.

In some embodiments, the process 80 can include receiving 132 first audio input. In some embodiments, the process 80 can include performing 134 speech recognition on the first audio input to generate a first text output based on the first audio input. In some embodiments, the process 80 can include determining 136 if a single first independent clause is included in the first text output.

If the first text output is determined to include a single independent clause, the process 80 can include determining 140 if the first text output includes any adverbial phrase or any prepositional phrase outside the single first independent clause. Otherwise, if the first text output is determined not to include a single independent clause (e.g., if the first text output is determined to include no independent clauses or more than one independent clause), the process 80 can include generating 138 an error notification. The error notification can indicate that the first text output does not include the single first independent clause. The error notification is intended to notify a client that their audio input is overly complex, and that the client should simplify their utterances.

If the first text output is determined not to include any adverbial phrase or any prepositional phrase outside the single first independent clause, the process 80 can include generating 142 a first symbol having a first predetermined size and a first predetermined shape at approximately a first location on a user interface.

Any additional symbol can be generated having another predetermined size and another predetermined shape. Each additional symbol can be generated at an additional location, wherein the additional location is positioned generally along a predetermined direction from the a previous location of a previously generated symbol.

If the first text output is determined to include any adverbial phrase or any prepositional phrase outside the single first independent clause, the process 80 can include generating 138 an error notification. The error notification can indicate that at least one adverbial phrase or prepositional phrase is outside the single first independent clause.

FIG. 4B illustrates a network diagram of a system 200 including detailed features of one or more computing devices 102, according to some embodiments disclosed herein. The example system 200 includes the one or more computer devices 102 connected to at least one user device (e.g., 108, 110, 112 in FIG. 1A) to receive user audio input 201. The user audio input 201 can be received via one or more microphones of the user device.

The one or more computer devices 102 can be configured to host an artificial intelligence/machine learning (Al/ML) model 107. The one or more computer devices 102 can receive user audio input provided by a user device and historical textual data retrieved from one or more databases. The historical textual data can include any publicly available data.

It should be understood that the one or more computing devices 102 may include additional components and that some of the components described herein may be removed and/or modified without departing from a scope of the one or more computing devices 102 disclosed herein. The one or more computing devices 102 may include one or more processors 204, which may include a semiconductor-based microprocessor, a central processing unit (CPU), an application specific integrated circuit (ASIC), a field programmable gate array (FPGA), and/or another hardware device. Although a single processor 204 is depicted, it should be understood that the one or more computing devices 102 may include multiple processors, multiple cores, or the like, without departing from the scope of the one or more computing devices 102.

The one or more computing devices 102 may also include a non-transitory computer readable medium 212 that may have stored thereon machine-readable instructions executable by the one or more processors 204. Examples of the machine-readable instructions are shown as 214-228 and are further discussed below. Examples of the non transitory computer readable medium 212 may include an electronic, magnetic, optical, or other physical storage device that contains or stores executable instructions. For example, the non-transitory computer readable medium 212 may include random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), any other form of storage medium known in the art, or any combination thereof.

The one or more processors 204 may fetch, decode, and execute the machine-readable instructions 214 to receive first audio input. The one or more processors 204 may fetch, decode, and execute the machine-readable instructions 216 to perform speech recognition on the first audio input to generate a first text output based on the first audio input.

The one or more processors 204 may fetch, decode, and execute the machine-readable instructions 218 to apply natural language processing to the first text output to generate grammatical analysis of the first text output. For example, the one or more processors 204 may fetch, decode, and execute the machine-readable instructions 220 to determine if a single first independent clause is included in the first text output. The one or more processors 204 may fetch, decode, and execute the machine-readable instructions 222 to determine if the first text output includes any adverbial phrase or any prepositional phrase outside the single first independent clause.

The one or more processors 204 may fetch, decode, and execute the machine-readable instructions 224 to perform any additional grammatical analysis on the first text output. For example, the one or more processors 204 may fetch, decode, and execute the machine readable instructions 224 to determine if the first text output includes an idiom. The one or more processors 204 may fetch, decode, and execute the machine-readable instructions 224 to determine if the first text output includes any conjunction. The one or more processors 204 may fetch, decode, and execute the machine-readable instructions 224 to determine if the first text output includes any conjunction outside the idiom. The one or more processors 204 may fetch, decode, and execute the machine-readable instructions 224 to determine if the first text output includes a predetermined number of verbs. The one or more processors 204 may fetch, decode, and execute the machine-readable instructions 224 to determine if the first text output includes a single compound verb. The one or more processors 204 may fetch, decode, and execute the machine-readable instructions 224 to determine if the first text output includes any verb outside the single compound verb. The one or more processors 204 may fetch, decode, and execute the machine-readable instructions 224 to determine if the first text output includes a single verb. The one or more processors 204 may fetch, decode, and execute the machine-readable instructions 224 to determine if the first text output includes a predetermined number of words. The one or more processors 204 may fetch, decode, and execute the machine-readable instructions 224 to determine if the first text output includes an adjective phrase, an infinitive phrase, a participial phrase, a gerundial phrase, a dependent clause, any other suitable clause(s), any other suitable phrase(s), or any combination thereof, outside the single first independent clause.

The one or more processors 204 may fetch, decode, and execute the machine-readable instructions 226 to generate a first symbol having a first predetermined size and a first predetermined shape at approximately a first location on a user interface. The one or more processors 204 may fetch, decode, and execute the machine-readable instructions 228 to generate a next symbol having another predetermined size and another predetermined shape at approximately another location that is within a predetermined distance from the location of the previously generated symbol.

The AI/ML module 107 may generate a predictive model(s) based at least on the user audio input 201, historical textual data, or a combination thereof. In one embodiment, the user audio input 201 and the historical textual data may be normalized and standardized by a data normalization engine (not shown). The AI/ML module 107 may provide predictive outputs data in the form of generative text parameters for grammatical analysis of the user audio input. The one or more computing devices 102 may process the predictive outputs data received from the AI/ML model 107 to perform grammatical analysis. In one embodiment, the one or more computing devices 102 may acquire user audio input data from user devices continuously or periodically in order to check if a new generative text parameter needs to be generated.

The one or more processors 204 may fetch, decode, and execute the machine-readable instructions to train the AI/ML model 107. In some embodiments, the AI/ML model 107 may use training data sets to improve accuracy of the prediction of the generative text parameters. The generative text parameters used in training data sets may be stored in a centralized database or a decentralized database. In some embodiments, a neural network and a language model (e.g., a large language model) may be used in the AI/ML model 107 for generating and predicting generative text parameters.

Furthermore, training of the AI/ML model 107 on the audio input data and/or speech recognition data may take rounds of refinement and testing by the one or more computing devices 102. Each round may be based on additional data or data that was not previously considered to help expand the knowledge of the AI/ML model 107. Different training and testing steps (and the data associated therewith) may be stored by the one or more computing devices 102. Each refinement of the AI/ML model 107 (e.g., changes in variables, weights, etc.) may be stored by the one or more computing devices 102. After the model has been trained, it may be deployed to a live environment where it can generate grammatical analysis based on the execution of the final trained machine learning model using the generative text parameters as part of the AI/ML model 107.

Referring to FIG. 4C, a user interface 400 of a user device (e.g., 108, 110, 112 in FIG. 1A) can be generated by a system (e.g., 100 in FIG. 1A). When first audio input is received for grammatical analysis, a first symbol 161 can be generated at a first location on the user interface 400. When second audio input is received for grammatical analysis, a second symbol 162 can be generated at a second location on the user interface 400. The second symbol 162 can be generated proximate to the first symbol 161 and along a predetermined direction 160 from the first symbol 161 on the user interface 400. When third audio input is received for grammatical analysis, a third symbol 163 can be generated at a third location on the user interface 400. The third symbol 163 can be generated proximate to the second symbol 162 and along the predetermined direction 160 from the second symbol 162 on the user interface 400. The first symbol 161, the second symbol 162, and the third symbol 163 can be generated along a non-intersecting path (e.g., 70 in FIG. 3B).

To create one or more notes, a recording icon 402 can be selected to initiate recording of user audio input and to generate output text 404 that can be associated with the next or last generated symbol (e.g., 161, 162, 163). Upon selecting a symbol (e.g., 161, 162, 163) associated with output text 404, an effect can be applied to the symbol and the output text such as, for example, highlighting, color changing, etc. so that a client can associate a symbol with a respective output text. In some embodiments, a client can type text as notes rather than by recording user audio input.

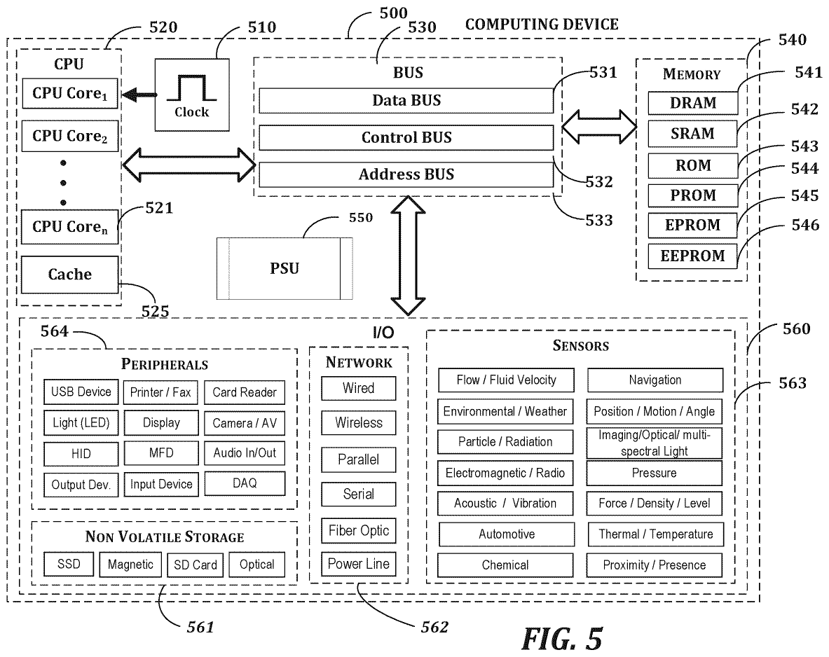

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative embodiment, the processor and the storage medium may reside as discrete components. For example, FIG. 5 illustrates an example computing device (e.g., the one or more computing devices, the one or more user devices 106 in FIG. 1A) 500, which may represent or be integrated in any of the above-described components, etc.

FIG. 5 illustrates a block diagram of a system including computing device 500. The computing device 500 may comprise, but not be limited to the following:

Mobile computing device, such as, but is not limited to, a laptop, a tablet, a smartphone, a drone, a wearable, an embedded device, a handheld device, an Arduino, an industrial device, or a remotely operable recording device;

A supercomputer, an exa-scale supercomputer, a mainframe, or a quantum computer;

A minicomputer, wherein the minicomputer computing device comprises, but is not limited to, an IBM AS500/iSeries/System I, A DEC VAX/PDP, a HP3000, a Honeywell Bull DPS, a Texas Instruments TI-990, or a Wang Laboratories VS Series;

A microcomputer, wherein the microcomputer computing device comprises, but is not limited to, a server, wherein a server may be rack mounted, a workstation, an industrial device, a raspberry pi, a desktop, or an embedded device;

Embodiments of the present disclosure may comprise a computing device having a central processing unit (CPU) 520, a bus 530, a memory unit 550, a power supply unit (PSU) 550, and one or more Input/Output (1/0) units. The CPU 520 coupled to the memory unit 550 and the plurality of 1/0 units 560 via the bus 530, all of which are powered by the PSU 550. It should be understood that, in some embodiments, each disclosed unit may actually be a plurality of such units for the purposes of redundancy, high availability, and/or performance. The combination of the presently disclosed units is configured to perform the stages of any method disclosed herein.

Consistent with an embodiment of the disclosure, the aforementioned CPU 520, the bus 530, the memory unit 550, a PSU 550, and the plurality of 1/0 units 560 may be implemented in a computing device, such as computing device 500. Any suitable combination of hardware, software, or firmware may be used to implement the aforementioned units. For example, the CPU 520, the bus 530, and the memory unit 550 may be implemented with computing device 500 or any of other computing devices 500, in combination with computing device 500. The aforementioned system, device, and components are examples and other systems, devices, and components may comprise the aforementioned CPU 520, the bus 530, the memory unit 550, consistent with embodiments of the disclosure.

At least one computing device 500 may be embodied as any of the computing elements illustrated in all of the attached figures, including the one or more computing devices 102 (FIG. 1A). A computing device 500 does not need to be electronic, nor even have a CPU 520, nor bus 530, nor memory unit 550.

With reference to FIG. 5, a system consistent with an embodiment of the disclosure may include a computing device, such as computing device 500. In a basic configuration, computing device 500 may include at least one clock module 510, at least one CPU 520, at least one bus 530, and at least one memory unit 550, at least one PSU 550, and at least one 1/0 560 module, wherein 1/0 module may be comprised of, but not limited to a non-volatile storage sub-module 561, a communication sub-module 562, a sensors sub-module 563, and a peripherals sub-module 565.

A system consistent with an embodiment of the disclosure the computing device 500 may include the clock module 510 may be known to a person having ordinary skill in the art as a clock generator, which produces clock signals. Clock signal is a particular type of signal that oscillates between a high and a low state and is used like a metronome to coordinate actions of digital circuits. Most integrated circuits (ICs) of sufficient complexity use a clock signal in order to synchronize different parts of the circuit, cycling at a rate slower than the worst-case internal propagation delays. The preeminent example of the aforementioned integrated circuit is the CPU 520, the central component of modern computers, which relies on a clock. The only exceptions are asynchronous circuits such as asynchronous CPUs. The clock 510 can comprise a plurality of embodiments, such as, but not limited to, single-phase clock which transmits all clock signals on effectively 1 wire, two-phase clock which distributes clock signals on two wires, each with non-overlapping pulses, and four-phase clock which distributes clock signals on 5 wires.

Many computing devices 500 use a “clock multiplier” which multiplies a lower frequency external clock to the appropriate clock rate of the CPU 520. This allows the CPU 520 to operate at a much higher frequency than the rest of the computer, which affords performance gains in situations where the CPU 520 does not need to wait on an external factor (like memory 550 or input/output 560). Some embodiments of the clock 510 may include dynamic frequency change, where the time between clock edges can vary widely from one edge to the next and back again.

A system consistent with an embodiment of the disclosure the computing device 500 may include the CPU unit 520 comprising at least one CPU Core 521. A plurality of CPU cores 521 may comprise identical CPU cores 521, such as, but not limited to, homogeneous multi-core systems. It is also possible for the plurality of CPU cores 521 to comprise different CPU cores 521, such as, but not limited to, heterogeneous multi-core systems, big. LITTLE systems and some AMD accelerated processing units (APU). The CPU unit 520 reads and executes program instructions which may be used across many application domains, for example, but not limited to, general purpose computing, embedded computing, network computing, digital signal processing (DSP), and graphics processing (GPU). The CPU unit 520 may run multiple instructions on separate CPU cores 521 at the same time. The CPU unit 520 may be integrated into at least one of a single integrated circuit die and multiple dies in a single chip package. The single integrated circuit die and multiple dies in a single chip package may contain a plurality of other aspects of the computing device 500, for example, but not limited to, the clock 510, the CPU 520, the bus 530, the memory 550, and 1/0 560.

The CPU unit 520 may contain cache 522 such as, but not limited to, a level 1 cache, level 2 cache, level 3 cache or combination thereof. The aforementioned cache 522 may or may not be shared amongst a plurality of CPU cores 521. The cache 522 sharing comprises at least one of message passing and inter-core communication methods may be used for the at least one CPU Core 521 to communicate with the cache 522. The inter-core communication methods may comprise, but not limited to, bus, ring, two-dimensional mesh, and crossbar. The aforementioned CPU unit 520 may employ symmetric multiprocessing (SMP) design.

The plurality of the aforementioned CPU cores 521 may comprise soft microprocessor cores on a single field programmable gate array (FPGA), such as semiconductor intellectual property cores (IP Core). The plurality of CPU cores 521 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 521, for example, but not limited to Instruction-level parallelism (ILP) such as, but not limited to, superscalar pipelining, and Thread-level parallelism (TLP).

Consistent with the embodiments of the present disclosure, the aforementioned computing device 500 may employ a communication system that transfers data between components inside the aforementioned computing device 500, and/or the plurality of computing devices 500. The aforementioned communication system will be known to a person having ordinary skill in the art as a bus 530. The bus 530 may embody internal and/or external plurality of hardware and software components, for example, but not limited to a wire, optical fiber, communication protocols, and any physical arrangement that provides the same logical function as a parallel electrical bus. The bus 530 may comprise at least one of, but not limited to a parallel bus, wherein the parallel bus carry data words in parallel on multiple wires, and a serial bus, wherein the serial bus carry data in bit-serial form. The bus 530 may embody a plurality of topologies, for example, but not limited to, a multidrop / electrical parallel topology, a daisy chain topology, and a connected by switched hubs, such as USB bus. The bus 530 may comprise a plurality of embodiments, for example, but not limited to:
Internal data bus (data bus) 531/Memory bus
Control bus 532
Address bus 533
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface(MIPI)
Coherent Processor Interface (CAPI)
Plug-n-play
1-Wire
Peripheral Component Interconnect (PCI), including embodiments such as, but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCie v2], PCI Express External Cabling [ePCie], and PCI Express OCuLink [Optical Copper{Cu} Link]), Express Card, AdvancedTCA, AMC, Universal IO, Thunderbolt/Mini DisplayPort, Mobile PCie (M-PCle), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/105 bus (e.g., PC/105-Plus, PCI/105-Express, PCI/105, and PCI-105), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1395 Interface/Firewire, Thunderbolt, and eXtensible Host Controller Interface (xHCI).

Consistent with the embodiments of the present disclosure, the aforementioned computing device 500 may employ hardware integrated circuits that store information for immediate use in the computing device 500, known to the person having ordinary skill in the art as primary storage or memory 550. The memory 550 operates at high speed, distinguishing it from the non-volatile storage sub-module 561, which may be referred to as secondary or tertiary storage, which provides slow-to-access information but offers higher capacities at lower cost. The contents contained in memory 550, may be transferred to secondary storage via techniques such as, but not limited to, virtual memory and swap. The memory 550 may be associated with addressable semiconductor memory, such as integrated circuits consisting of silicon-based transistors, used for example as primary storage but also other purposes in the computing device 500. The memory 550 may comprise a plurality of embodiments, such as, but not limited to volatile memory, non-volatile memory, and semi volatile memory. It should be understood by a person having ordinary skill in the art that the ensuing are non-limiting examples of the aforementioned memory:
Volatile memory which requires power to maintain stored information, for example, but not limited to, Dynamic Random-Access Memory (DRAM) 551, Static Random-Access Memory (SRAM) 552, CPU Cache memory 525, Advanced Random-Access Memory (A RAM), and other types of primary storage such as Random-Access Memory (RAM).
Non-volatile memory which can retain stored information even after power is removed, for example, but not limited to, Read-Only Memory (ROM) 553, Programmable ROM (PROM) 555, Erasable PROM (EPROM) 555, Electrically Erasable PROM (EEPROM) 556 (e.g., flash memory and Electrically Alterable PROM [EAPROM]), Mask ROM (MROM), One Time Programmable (OTP) ROM I Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi volatile memory may comprise volatile and non-volatile memory and/or volatile memory with battery to provide power after power is removed. The semi-volatile memory may comprise, but not limited to spin-transfer torque RAM (STT-RAM).
Consistent with the embodiments of the present disclosure, the aforementioned computing device 500 may employ the communication system between an information processing system, such as the computing device 500, and the outside world, for example, but not limited to, human, environment, and another computing device 500. The aforementioned communication system will be known to a person having ordinary skill in the art as 1/0 560. The 1/0 module 560 regulates a plurality of inputs and outputs with regard to the computing device 500, wherein the inputs are a plurality of signals and data received by the computing device 500, and the outputs are the plurality of signals and data sent from the computing device 500. The 1/0 module 560 interfaces a plurality of hardware, such as, but not limited to, non-volatile storage 561, communication devices 562, sensors 563, and peripherals 565. The plurality of hardware is used by at least one of, but not limited to, human, environment, and another computing device 500 to communicate with the present computing device 500. The 1/0 module 560 may comprise a plurality of forms, for example, but not limited to channel 1/0, port mapped 1/0, asynchronous 1/0, and Direct Memory Access (DMA).
Consistent with the embodiments of the present disclosure, the aforementioned computing device 500 may employ the non-volatile storage sub-module 561, which may be referred to by a person having ordinary skill in the art as one of secondary storage, external memory, tertiary storage, off-line storage, and auxiliary storage. The non-volatile storage sub-module 561 may not be accessed directly by the CPU 520 without using an intermediate area in the memory 550. The non-volatile storage sub-module 561 does not lose data when power is removed and may be two orders of magnitude less costly than storage used in memory modules, at the expense of speed and latency. The non-volatile storage sub-module 561 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (561) may comprise a plurality of embodiments, such as, but not limited to:
Optical storage, for example, but not limited to, Compact Disk (CD) (CD-ROM/CD-RI CD-RW), Digital Versatile Disk (DVD) (DVD-ROM/DVD-RI DVD+R I DVD-RW/DVD+RW/DVD±RW/DVD+R DL/DVD-RAM/HD-DVD), Blu-ray Disk (BD) (BO ROM I BD-R I BD-RE I BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the aforementioned computing device 500 may employ the communication sub-module 562 as a subset of the 1/0 560, which may be referred to by a person having ordinary skill in the art as at least one of, but not limited to, computer network, data network, and network. The network allows computing devices 500 to exchange data using connections, which may be known to a person having ordinary skill in the art as data links, between network nodes. The nodes comprise network computer devices 500 that originate, route, and terminate data. The nodes are identified by network addresses and can include a plurality of hosts consistent with the embodiments of a computing device 500. The aforementioned embodiments include, but not limited to personal computers, phones, servers, drones, and networking devices such as, but not limited to, hubs, switches, routers, modems, and firewalls.

Two nodes can be networked together, when one computing device 500 is able to exchange information with the other computing device 500, whether or not they have a direct connection with each other. The communication sub-module 562 supports a plurality of applications and services, such as, but not limited to World Wide Web (WWW), digital video and audio, shared use of application and storage computing devices 500, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 5[1Pv 5 ], and Internet Protocol version 6[1Pv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [IDEN]).

The communication sub-module 562 may comprise a plurality of size, topology, traffic control mechanism and organizational intent. The communication sub-module 562 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Cellular systems embody technologies such as, but not limited to, 3G,5G (such as WiMax and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line and wireless communications

The aforementioned network may comprise a plurality of layouts, such as, but not limited to, bus network such as ethemet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 500 may employ the sensors sub-module 563 as a subset of the 1/0 560. The sensors sub-module 563 comprises at least one of the devices, modules, and subsystems whose purpose is to detect events or changes in its environment and send the information to the computing device 500. Sensors are sensitive to the measured property, are not sensitive to any property not measured, but may be encountered in its application, and do not significantly influence the measured property. The sensors sub-module 563 may comprise a plurality of digital devices and analog devices, wherein if an analog device is used, an Analog to Digital (A-to-D) converter must be employed to interface the said device with the computing device 500. The sensors may be subject to a plurality of deviations that limit sensor accuracy. The sensors sub-module 563 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:

Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nano-sensors).

Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensor (guitar pickup), seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermos-luminescent dosimeter.
Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, LiDAR, multi-spectral light sensor, colorimeter, contact image sensor, electro-optical sensor, infra-red sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photo-switch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezo capacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared I quartz I resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 500 may employ the peripherals sub-module 562 as a subset of the 1/0 560. The peripheral sub-module 565 comprises ancillary devices used to put information into and get information out of the computing device 500. There are 3 categories of devices comprising the peripheral sub-module 565, which exist based on their relationship with the computing device 500, input devices, output devices, and input/output devices. Input devices send at least one of data and instructions to the computing device 500. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to position of a mouse.
The number of degrees of freedom involved, such as, but not limited to, two dimensional mice vs three-dimensional mice used for Computer-Aided Design (CAD) applications.

Output devices provide output from the computing device 500. Output devices convert electronically generated information into a form that can be presented to humans. Input/output devices that perform both input and output functions. It should be understood by a person having ordinary skill in the art that the ensuing are non-limiting embodiments of the aforementioned peripheral sub-module 565:

### Input Devices

Human Interface Devices (HID), such as, but not limited to, pointing device (e.g., mouse, touchpad, joystick, touchscreen, game controller I gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).
High degree of freedom devices, that require up to six degrees of freedom such as, but not limited to, camera gimbals, Cave Automatic Virtual Environment (CAVE), and virtual reality systems.
Video Input devices are used to digitize images or video from the outside world into the computing device 500. The information can be stored in a multitude of formats depending on the user's requirement. Examples of types of video input devices include, but not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
Audio input devices are used to capture sound. In some cases, an audio output device can be used as an input device, in order to capture produced sound. Audio input devices allow a user to send audio signals to the computing device 500 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrument Digital Interface (MIDI) devices such as, but not limited to a keyboard, and headset.
Data Acquisition (DAQ) devices convert at least one of analog signals and physical parameters to digital values for processing by the computing device 500. Examples of DAQ devices may include, but not limited to, Analog to Digital Converter (ADC), data logger, signal conditioning circuitry, multiplexer, and Time to Digital Converter (TDC).

Output Devices may further comprise, but not be limited to:
Display devices, which convert electrical information into visual form, such as, but not limited to, monitor, TV, projector, and Computer Output Microfilm (COM). Display devices can use a plurality of underlying technologies, such as, but not limited to, Cathode Ray Tube (CRT), Thin-Film Transistor (TFT), Liquid Crystal Display (LCD), Organic Light Emitting Diode (OLED), MicroLED, E Ink Display (ePaper) and Refreshable Braille Display (Braille Terminal).

Printers, such as, but not limited to, inkjet printers, laser printers, 3D printers, solid ink printers and plotters.
Audio and Video (AV) devices, such as, but not limited to, speakers, headphones, amplifiers and lights, which include lamps, strobes, DJ lighting, stage lighting, architectural lighting, special effect lighting, and lasers.
Other devices such as Digital to Analog Converter (DAC)

Input/Output Devices may further comprise, but not be limited to, touchscreens, networking device (e.g., devices disclosed in network 562 sub-module), data storage device (non-volatile storage 561), facsimile (FAX), and graphics/sound cards.

The following description of variants is only illustrative of components, elements, acts, systems, media, and methods considered to be within the scope of the invention and are not in any way intended to limit such scope by what is specifically disclosed or not expressly set forth. The components, elements, acts, systems, media, and methods as described herein may be combined and rearranged other than as expressly described herein and are still considered to be within the scope of the invention.

According to variation 1, a method for performing cognitive-based therapy, can include performing, by a coach, a process to be emulated, the process including: uttering a first single independent clause identifying a first feature in a target subject matter; drawing a first symbol after uttering the first single independent clause; uttering a second single independent clause identifying a second feature in the target subject matter; drawing a second symbol proximate to the first symbol and along a first non-intersecting path after uttering the second single independent clause; uttering a third single independent clause identifying a third feature in the target subject matter; drawing a third symbol proximate to the second symbol and along the first non-intersecting path after uttering the third single independent clause; instructing, by the coach, a client to identify first features in a target subject matter by emulating the process; instructing, by the coach, the client to draw all symbols along a second non-intersecting path before the client emulates the process.

Variation 2 can include the method of variation 1, wherein the method includes instructing the client to identify additional features in the target subject matter by uttering using fewer words for each of the additional features than were uttered when the client emulated the process.

Variation 3 can include the method of variation 1, wherein the method includes instructing the client to identify additional features in the target subject matter by uttering a single independent clause for each of the additional features in the target subject matter.

Variation 4 can include the method of variation 1, further comprising: causing a demonstration to be presented, wherein the demonstration includes a recording of: a coach performing the process to be emulated.

Variation 5 can include the method of variation 1, further comprising: recording a coach performing the emulation process.

According to variation 6 a method for performing cognitive-based therapy can include uttering a first single independent clause identifying a first feature in a target subject matter; drawing a first symbol after uttering the first single independent clause; uttering a second single independent clause identifying a second feature in the target subject matter; drawing a second symbol proximate to the first symbol and along a first non-intersecting path after uttering the second single independent clause; uttering a third single independent clause identifying a third feature in the target subject matter; drawing a third symbol proximate to the second symbol and along the first non-intersecting path after uttering the third single independent clause.

Variation 7 can include the method of variation 6, wherein the method includes instructing the client to identify features in the target subject matter by uttering for each of the features.

Variation 8 can include the method of variation 7, wherein the method includes instructing the client to identify additional features in the target subject matter by uttering a single independent clause for each of the additional features in the target subject matter.

Variation 9 can include the method of variation 6, further comprising: causing a demonstration to be presented, wherein the demonstration includes a recording of a coach performing the method.

Variation 10 can include the method of variation 6, further comprising: recording a coach performing the method.

According to variation 11, a method for performing cognitive-based therapy can include receiving first audio input; performing speech recognition on the first audio input to generate a first text output based on the first audio input; determining that a single first independent clause is included in the first text output; determining that the first text output does not include any adverbial phrase or any prepositional phrase outside the single first independent clause; in response to determining that the single first independent clause is included in the first text output and in response determining that the first text output does not include any adverbial phrase or any prepositional phrase outside the single first independent clause, generating a first symbol having a first predetermined size and a first predetermined shape at approximately a first location on a user interface; receiving second audio input; performing speech recognition on the second audio input to generate a second text output based on the second audio input; determining that a single second independent clause is included in the second text output; determining that the second text output includes a first adverbial phrase or a first prepositional phrase outside the single second independent clause; determine that the second text output does not include any other independent clause that includes the first adverbial phrase or the first prepositional phrase; in response to determining that the second text output includes the first adverbial phrase or the first prepositional phrase outside the single second independent clause and in response to determining that the second text output does not include any other independent clause that includes the first adverbial phrase or the first prepositional phrase, generating a first error notification on the user interface, the error notification indicating that the second text output includes the first adverbial phrase or the first prepositional phrase outside the single second independent clause; receiving third audio input; performing speech recognition on the third audio input to generate a third text output based on the third audio input; determining that a single third independent clause is included in the third text output; determining that the third text output does not include any adverbial phrase or any prepositional phrase outside the single third independent clause; in response to determining that the third text output does not include any adverbial phrase or any prepositional phrase outside the single third independent clause, generating a second symbol having a second predetermined size and a second predetermined shape at approximately a second location proximate to the first location on the user interface, the second location being positioned generally along a predetermined direction from the first location on the user interface.

Variation 12 can include the method of variation 11, further comprising: storing an association between the first text output and the first symbol.

Variation 13 can include the method of variation 11, further comprising: determining that the first text output includes an idiom; determining that the first text output does not include any conjunction outside the idiom.

Variation 14 can include the method of variation 11, further comprising: determining that the first text output does not include an idiom; determining that the first text output does not include any conjunction.

Variation 15 can include the method of variation 11, further comprising: determining that the first text output does not include any conjunction.

Variation 16 can include the method of variation 11, further comprising: determining that the first text output includes a predetermined number of verbs.

Variation 17 can include the method of variation 11, further comprising: determining that the first text output includes a single compound verb; determining that the first text output does not include any verb outside the single compound verb.

Variation 18 can include the method of variation 11, further comprising: determining that the first text output does not include any compound verb; determining that the first text output includes a single verb.

Variation 19 can include the method of variation 11, further comprising: determining that the first text output includes a predetermined number of words.

According to variation 20, a system can include memory and one or more processors coupled to the memory, wherein the one or more processors are configured at least to perform the method of any one of variations 11-19.

According to variation 21, a non-transitory computer-readable medium can include instructions, that when executed by one or more processors, cause the one or more processors to perform the method of any one of variations 11-19.

According to variation 21, the method of any one of variations 11-19 is a computer implemented method.

It will be understood that it would be unduly repetitious and obfuscating to describe and illustrate every reordering, combination and subcombination of the elements and the aspects described. Accordingly, all elements, processes, and subprocesses can be combined in any way and/or combination, and the present specification, including the drawings, shall be construed to constitute a complete written description of all reorderings, combinations and subcombinations of the elements, processes, and subprocesses and of the aspects described herein, and of the manner and process of making and using the elements, and shall support claims to any such combination or subcombination. Any processes and subprocesses disclosed herein can be performed in any suitable order.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of’).

What is claimed is:

1. A computer-implemented method for providing cognitive-based therapy via a networked computing system, the method comprising:
receiving, by one or more processors of a server computing device from a client computing device over a communication network, a first audio input captured by a microphone of the client computing device during a cognitive therapy session;
performing, by the one or more processors, automatic speech recognition on the first audio input to generate a first text output;
applying, by the one or more processors, natural language processing to the first text output to perform a grammatical analysis, wherein the grammatical analysis comprises determining whether the first text output contains a single first independent clause and determining whether the first text output includes any adverbial phrase or any prepositional phrase outside the single first independent clause;
in response to determining that the first text output contains the single first independent clause and does not include any adverbial phrase or any prepositional phrase outside the single first independent clause, transmitting, by the one or more processors over the communication network to the client computing device, display instructions that cause the client computing device to render a first symbol having a first predetermined size and a first predetermined shape at a first location on a user interface displayed on a display of the client computing device;
receiving, by the one or more processors from the client computing device over the communication network, a second audio input captured by the microphone of the client computing device;
performing, by the one or more processors, automatic speech recognition on the second audio input to generate a second text output;
applying, by the one or more processors, natural language processing to the second text output to perform a second grammatical analysis, wherein the second grammatical analysis comprises determining whether the second text output contains a single second independent clause and determining whether the second text output includes a first adverbial phrase or a first prepositional phrase outside the single second independent clause;
in response to determining that the second text output includes the first adverbial phrase or the first prepositional phrase outside the single second independent clause, transmitting, by the one or more processors over the communication network to the client computing device, an error notification that causes the client computing device to display, on the user interface, an indication that the second text output includes the first adverbial phrase or the first prepositional phrase outside the single second independent clause;
receiving, by the one or more processors from the client computing device over the communication network, a third audio input captured by the microphone of the client computing device;
performing, by the one or more processors, automatic speech recognition on the third audio input to generate a third text output;
applying, by the one or more processors, natural language processing to the third text output to perform a third grammatical analysis; and
in response to the third grammatical analysis determining that the third text output contains a single third independent clause and does not include any adverbial phrase or any prepositional phrase outside the single third independent clause, transmitting, by the one or more processors over the communication network to the client computing device, second display instructions that cause the client computing device to render a second symbol having a second predetermined size and a second predetermined shape at a second location on the user interface, the second location being proximate to the first location and positioned along a predetermined non-intersecting path direction from the first location on the user interface.

2. The computer-implemented method of claim 1, further comprising:
storing, by the one or more processors in a database communicatively coupled to the server computing device, an association between the first text output and the first symbol, and an association between the third text output and the second symbol.

3. The computer-implemented method of claim 1, further comprising:
applying, by the one or more processors, natural language processing to the first text output to determine that the first text output includes an idiom; and
determining, by the one or more processors, that the first text output does not include any conjunction outside the idiom.

4. The computer-implemented method of claim 1, further comprising:
determining, by the one or more processors, that the first text output does not include an idiom; and
determining, by the one or more processors, that the first text output does not include any conjunction.

5. The computer-implemented method of claim 1, further comprising:
determining, by the one or more processors via the natural language processing, that the first text output includes a predetermined number of verbs.

6. The computer-implemented method of claim 1, further comprising:
determining, by the one or more processors, that the first text output includes a single compound verb; and
determining, by the one or more processors, that the first text output does not include any verb outside the single compound verb.

7. The computer-implemented method of claim 1, further comprising:
determining, by the one or more processors, that the first text output does not include any compound verb; and
determining, by the one or more processors, that the first text output includes a single verb.

8. The computer-implemented method of claim 1, further comprising:
determining, by the one or more processors, that the first text output includes a predetermined number of words.

9. The computer-implemented method of claim 1, wherein the natural language processing is performed by an artificial intelligence and machine learning model hosted on the server computing device, and the method further comprises:
training the artificial intelligence and machine learning model using historical textual data and previously received audio input data to generate generative text parameters for the grammatical analysis.

10. The computer-implemented method of claim 1, further comprising:
applying, by the one or more processors, natural language processing to the first text output to determine whether the first text output includes an adjective phrase, an infinitive phrase, a participial phrase, a gerundial phrase, or a dependent clause outside the single first independent clause.

11. A system for providing cognitive-based therapy, the system comprising:
a communication network;
a client computing device comprising a microphone, a display, and a client processor, the client computing device communicatively coupled to the communication network;
a server computing device comprising one or more server processors and a non-transitory computer-readable memory storing instructions that, when executed by the one or more server processors, cause the one or more server processors to:
receive, from the client computing device over the communication network, a first audio input captured by the microphone of the client computing device during a cognitive therapy session;
perform automatic speech recognition on the first audio input to generate a first text output;
apply natural language processing to the first text output to perform a grammatical analysis comprising determining whether the first text output contains a single first independent clause and determining whether the first text output includes any adverbial phrase or any prepositional phrase outside the single first independent clause;
in response to determining that the first text output contains the single first independent clause and does not include any adverbial phrase or any prepositional phrase outside the single first independent clause, transmit, over the communication network to the client computing device, display instructions that cause the client computing device to render a first symbol having a first predetermined size and a first predetermined shape at a first location on a user interface displayed on the display of the client computing device;
receive, from the client computing device over the communication network, a second audio input captured by the microphone of the client computing device;
perform automatic speech recognition on the second audio input to generate a second text output;
apply natural language processing to the second text output to determine whether the second text output includes a first adverbial phrase or a first prepositional phrase outside a single second independent clause;
in response to determining that the second text output includes the first adverbial phrase or the first prepositional phrase outside the single second independent clause, transmit, over the communication network to the client computing device, an error notification that causes the client computing device to display an indication that the second text output includes the first adverbial phrase or the first prepositional phrase outside the single second independent clause;
receive, from the client computing device over the communication network, a third audio input;
perform automatic speech recognition on the third audio input to generate a third text output;
apply natural language processing to the third text output to perform a third grammatical analysis; and
in response to the third grammatical analysis determining that the third text output contains a single third independent clause and does not include any adverbial phrase or any prepositional phrase outside the single third independent clause, transmit, over the communication network to the client computing device, second display instructions that cause the client computing device to render a second symbol having a second predetermined size and a second predetermined shape at a second location on the user interface, the second location being proximate to the first location and positioned along a predetermined non-intersecting path direction from the first location on the user interface.

12. The system of claim 11, wherein the instructions further cause the one or more server processors to:
store, in a database communicatively coupled to the server computing device, an association between the first text output and the first symbol, and an association between the third text output and the second symbol.

13. The system of claim 11, wherein the instructions further cause the one or more server processors to:
apply natural language processing to the first text output to determine that the first text output includes an idiom; and
determine that the first text output does not include any conjunction outside the idiom.

14. The system of claim 11, wherein the instructions further cause the one or more server processors to:
determine that the first text output includes a single compound verb and does not include any verb outside the single compound verb.

15. The system of claim 11, wherein the natural language processing is performed by an artificial intelligence and machine learning model hosted on the server computing device, the artificial intelligence and machine learning model trained using historical textual data and previously received audio input data to generate generative text parameters for the grammatical analysis.

16. A non-transitory computer-readable medium storing instructions that, when executed by one or more processors of a server computing device communicatively coupled to a client computing device over a communication network, cause the one or more processors to:
receive, from the client computing device over the communication network, a first audio input captured by a microphone of the client computing device during a cognitive therapy session;
perform automatic speech recognition on the first audio input to generate a first text output;
apply natural language processing to the first text output to perform a grammatical analysis comprising determining whether the first text output contains a single first independent clause and determining whether the first text output includes any adverbial phrase or any prepositional phrase outside the single first independent clause;
in response to determining that the first text output contains the single first independent clause and does not include any adverbial phrase or any prepositional phrase outside the single first independent clause, transmit, over the communication network to the client computing device, display instructions that cause the client computing device to render a first symbol having a first predetermined size and a first predetermined shape at a first location on a user interface displayed on a display of the client computing device;
receive, from the client computing device over the communication network, a subsequent audio input;
perform automatic speech recognition on the subsequent audio input to generate a subsequent text output;
apply natural language processing to the subsequent text output to perform a subsequent grammatical analysis; and
based on results of the subsequent grammatical analysis, selectively transmit, over the communication network to the client computing device, either second display instructions that cause the client computing device to render a second symbol at a second location proximate to the first location and positioned along a predetermined non-intersecting path direction from the first location on the user interface, or an error notification that causes the client computing device to display an indication that the subsequent text output does not conform to a required grammatical structure.

17. The non-transitory computer-readable medium of claim 16, wherein the instructions further cause the one or more processors to:
store, in a database, an association between the first text output and the first symbol.

18. The non-transitory computer-readable medium of claim 16, wherein the grammatical analysis further comprises determining whether the first text output includes an idiom and determining whether the first text output includes any conjunction outside the idiom.

19. The non-transitory computer-readable medium of claim 16, wherein the grammatical analysis further comprises determining whether the first text output includes a predetermined number of words.

20. The non-transitory computer-readable medium of claim 16, wherein the natural language processing is performed by an artificial intelligence and machine learning model hosted on the server computing device, the artificial intelligence and machine learning model trained using historical textual data to generate generative text parameters for the grammatical analysis.
